(19) United States
(12) Patent Application Publication
(10) Pub. No.: US 2017/0177815 A1
(43) Pub. Date: Jun. 22, 2017

(54) WEARABLE HEALTH MESSAGE DELIVERY SYSTEM WITH CO EXPOSURE LIMITER

(71) Applicant: Intel Corporation, Santa Clara, CA (US)

(72) Inventors: Narayan Sundararajan, Palo Alto, CA (US); Lakshman Krishnamurthy, Portland, OR (US); Sabar Mourad Souag, Beaverton, OR (US); Braxton Lathrop, Lake Oswego, OR (US); Cynthia Lynn Merrill, Portland, OR (US)

(21) Appl. No.: 14/979,324

(22) Filed: Dec. 22, 2015

(51) Int. Cl. G06F 19/00; G08B 21/14; G01N 33/00

(52) U.S. Cl. CPC G06F 19/3406; G01N 33/0063; G08B 21/14

(57) ABSTRACT

Embodiments of the present disclosure are directed to a culturally relevant wearable device that includes a carbon monoxide detector to detect carbon monoxide, a light emitting diode (LED), a memory for storing data; and a processor. The processor is configured to determine that a carbon monoxide (CO) level detected by the carbon monoxide detector exceeds a threshold level; and activate the LED based on the determination that the CO level detected by the CO detector exceeds the threshold level. The wearable device also includes a culturally relevant ornamental housing, the ornamental housing comprising a housing for the wearable device. The wearable device can also include a speaker to provide audible notifications about CO levels, maternity schedules, vaccination schedules, therapy schedules, and disease management schedules.

### TECHNICAL FIELD

This disclosure pertains to a wearable health message delivery system, and in particular, to a culturally relevant wearable health message delivery system that includes a carbon monoxide exposure limiter and a processor to provide medically pertinent alerts.

### BACKGROUND

Indoor air pollution is a major cause of illness and death in developing countries with 4 million deaths annually associated with it. Indoor air pollution is primarily caused by cooking using wood, charcoal, or cow dung as fuel. Carbon monoxide (CO), particulate matter <2.5 μm in size (PM2.5), and black carbon (soot) are the major components of the smoke caused by cooking using the above fuels. Women, particularly pregnant women and children, both born and unborn, form the highest risk population of the 4 million in danger. CO exposure, both acute and chronic, has short-term as well as long-term health effects such as neurological issues, birth defects, acute respiratory illness (ARI), chronic obstructive pulmonary disease (COPD), etc.

Also, maternal and child mortality in developing countries are still extremely high in spite of progress made towards the Millennium Development Goals 4 & 5. This is due to a variety of reasons, one of the key ones being lack of basic information to the women about their pregnancy, childbirth, and infant care.

The problem of indoor air pollution is being primarily tackled by efforts to design, build and distribute clean cook stoves which has been met with extremely limited success due to a variety of reasons. Efforts to provide relevant information to pregnant women has been ongoing with the mobile phones and associated delivery mechanism such as SMS and IVR being the primary technology option along with existing mobile health workers infrastructure. While this has met with some limited success, scale and sustainability remains an issue due to the cost of delivering the information and who pays for it.

### BRIEF DESCRIPTION OF THE DRAWINGS

FIG. 1 is a schematic block diagram of a carbon monoxide exposure limiter system in accordance with embodiments of the present disclosure.

FIG. 2 is a schematic diagram of a wearable device with a carbon monoxide exposure limiter in accordance with embodiments of the present disclosure.

FIG. 3 is a schematic diagram of a flex circuit that includes a carbon monoxide sensor in accordance with embodiments of the present disclosure.

FIG. 4 is a process flow diagram for alerting a wearer of a wearable carbon monoxide exposure limiter of a carbon monoxide level in accordance with embodiments of the present disclosure.

FIG. 5 is a process flow diagram for providing an emergency alerting accordance with embodiments of the present disclosure.

FIG. 6 is a process flow diagram for providing maternity messages to a wearer of a wearable device including a carbon monoxide exposure limiter.

### DETAILED DESCRIPTION

This disclosure describes a culturally relevant wearable accessory for women that includes a carbon monoxide exposure limiter (COEL) that is able to provide an alarm based on CO exposure (and potentially PM2.5 exposure) as well as deliver pre-programmed tips, alerts, and information about medically pertinent information, such as information about pregnancy, childbirth, infant care, vaccine schedules, therapy schedules, and other medically pertinent information. In some implementations, the COEL can be solar-powered to reduce the dependence on consumable batteries.

This disclosure describes a device that takes the form of a culturally relevant accessory (bangle, necklace, chain etc.) and incorporates a CO sensor that is robust and sensitive, coupled with an output modality, such as a set of lights or a speaker, which is able to provide both alerts for CO alarms as well as information to the wearer about medically pertinent information, such as their pregnancy, childbirth, infant care, therapy or vaccination schedules, etc. The device is also powered by “hybrid” power sources of solar panels and a rechargeable battery such that there is no need to plug the device in or change batteries.

Currently CO monitoring is performed primarily as a research activity using bulky power hungry CO monitors with no feedback provided to the exposed individuals. Also, clean cook stoves that were supposed to address the root cause of the issue has not been very successful in terms of adoption due to multiple factors such as poor design and cost. Provision of information through SMS and IVR has faced scaling problems due to lack of interest from telecom providers, lack of mobile devices amongst women in rural areas, as well as high cost of delivering SMS/IVR messages in developing countries.

Advantages of this disclosure are directed to mitigating and addressing both these problems by designing a no-charging required wearable CO monitoring device that provides feedback as well as pre-programmed information relevant to pregnant women as one of the first focused use cases.

A wearable COEL device features and attributes are as follows:
Wearable and culturally relevant design for an accessory—necklace or a bracelet/bangle; Device detects CO levels on a daily basis and resets every day [Combo sensor for the math (humidity, temperature, pressure)]; Modular design to add more sensors later based on field-testing;
Audible alarm and speaker for voice alert that also provides reminders for milestones during pre- and post-pregnancy based on elapsed time; Acknowledgement feedback of voice reminder.
Color LED warnings, potentially low resolution display on band.
Ability to preprogram localized and customized voice alerts based on set times. Water resistant
Ability to transfer CO exposure and acknowledgement feedback data (USB, Bluetooth or other)
A battery that last for 10 months at the least. Energy harvesting with a mixed solution of solar charging+rechargeable battery, as well as power optimized circuitry Real-time digital clock

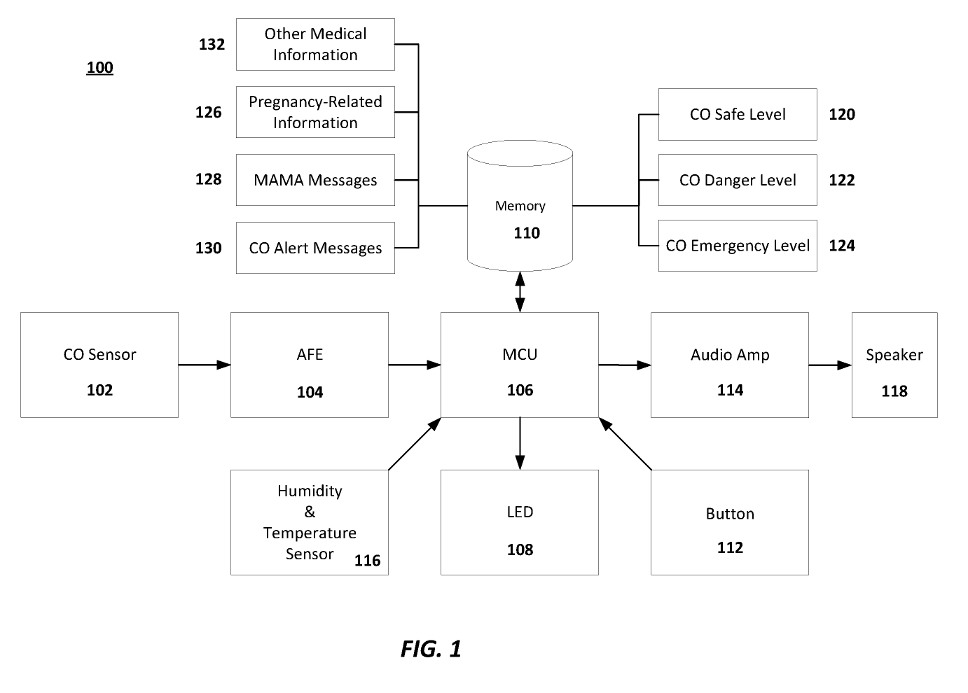

FIG. 1 is a schematic block diagram of a carbon monoxide (CO) exposure limiter (COEL) system 100 in accordance with embodiments of the present disclosure. In some embodiments, the COEL system 100 can be included in a wearable device, such as one that is culturally relevant for women in developing nations, the purpose being to allow for a COEL device on an unobtrusive wearable. The COEL system 100 includes a CO sensor 102, an analog front end (AFE) 104, a microprocessor unit (MCU) 106, and a memory 110. The CO sensor 102 can be implemented in hardware or a combination of hardware and software. The CO sensor 102 is configured to receive carbon monoxide from surrounding air and provide a signal to the AFE 104 representative of the CO level in the surrounding air. The CO sensor 102 can include an electrochemical detector, a semiconductor detector, an opto-chemical detector, biomimetic detector, etc. The CO sensor 102 can output an electrical signal representative of a CO level to an AFE 104 that can convert the electrical signal to a digital signal for input into the MCU 106.

The MCU 106 can process the received CO level signal to determine whether or how to indicate the CO level to a wearer of the wearable device. The COEL system 100 can include a memory 110. Memory 110 can store threshold CO levels for safe level 120, danger level 122, and emergency level 124. The MCU 106 can use the received CO level and compare the received CO level with the CO levels stored in memory. Table 1 and Table 2 provide examples of CO levels that can be considered safe, dangerous, or emergency levels.

The MCU 106 can activate a light emitting diode 108 based on the CO level detected. For example, a certain color LED can be activated based on the CO level. In some embodiments, an LED can flash at different periodicities to draw attention to emergency levels versus lower alert levels.

An LED 108 can also be used to indicate a lower battery level.

The memory 110 can also store CO level alert messages 130. The CO level alert messages 130 can be output by the MCU through a speaker 118. The MCU 106 can output a CO level alert message through an audio amplifier 114.

Additionally, the memory 110 can store pregnancy-related information 126. Pregnancy-related information 126 can include dates and events related to a pregnancy, such as due date, scheduled doctor's visits dates, etc. The memory 110 also stores Mobile Alliance for Maternal Action (MAMA) messages 128. MAMA messages 128 can be provided audibly to the wearer through the speaker based on a date or event for a pregnancy.

Additionally, memory 110 can store other medical information 132. Other medical information 132 can include messages that can instruct or inform the wearer about certain medical actions to be taken at certain times (e.g., based on time stamps). The other medical information 132 can include messages instructing the wearer about vaccine schedules, directly observed treatment short courses (DOTS), antiretroviral therapy scheduling (ARV), diabetes testing and shots, etc.

The MCU 106 can include a real-time clock that allows for precise timing for the delivery of messages. For example, the wearable can be programmed with a starting day and time. The wearable can also be programmed with scheduling information associated with pregnancy, disease control, therapy, etc. The MCU 106 can inform or instruct the wearer about the pregnancy schedule event based on the day and time.

The COEL system 100 can include a button 112. Button 112 can be used to request a CO level indication, program dates and times for alerts, resetting or overriding alerts, muting the messages, etc.

In some embodiments, the COEL system 100 can include a humidity and temperature sensor 116 for providing humidity and temperature information to the MCU 106. The MCU 106 can use the humidity and temperature information for accurate CO level measurements because, in some embodiments, the CO sensor readings change with temperature and humidity.

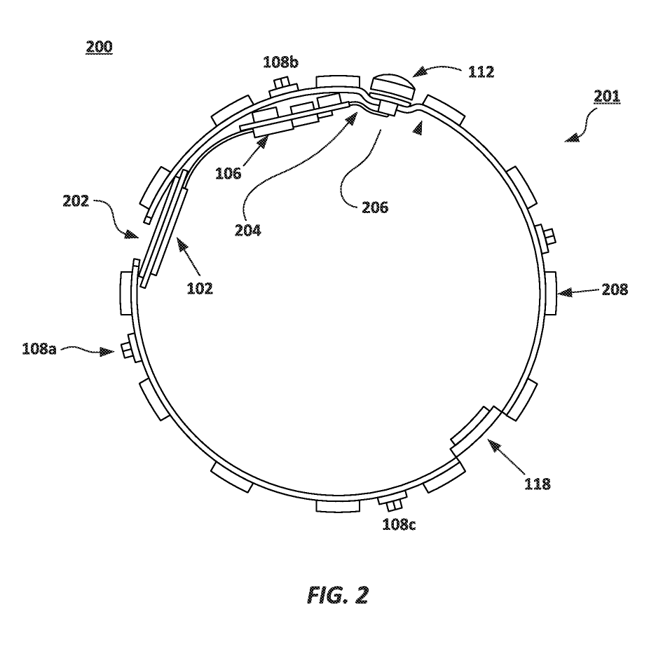

FIG. 2 is a schematic diagram of a wearable device 200 with a carbon monoxide exposure limiter in accordance with embodiments of the present disclosure. The wearable device 200 can be a culturally sensitive wearable, such as jewelry or other type of jewelry. The wearable device 200 includes printed circuit board (PCB) 201 that can link with an outer decorative portion. The PCB 201 can be a printed circuit board that holds/houses the electronic circuitry for monitoring CO levels and providing alerts.

The wearable device 200 shown includes a flex circuit that connects the CO sensor 102 to the microprocessing unit (MPU) 106. The PCB 201 can include a gap 202 for allowing air to contact the CO sensor 102. The wearable device 200 also includes a plurality of LEDs 108a, 108b, and 108c. Each LED can be connected to the MPU 106 to alert a wearer of the CO level. For example, each LED can include multiple colored LEDs, and the CO level can be used to activate different colored LED lights. The wearable device 200 also includes a button 112 that can be used by the wearer to request a CO level indication, as well as for other function.

The wearable device 200 also includes a speaker 118 for audibly providing the wearer with messages relating to CO levels (e.g., alerts, warnings, etc.) and pregnancy related information, such as MAMA messages.

The outer face of the PCB can include a plurality of solar cells 208 for powering the system.

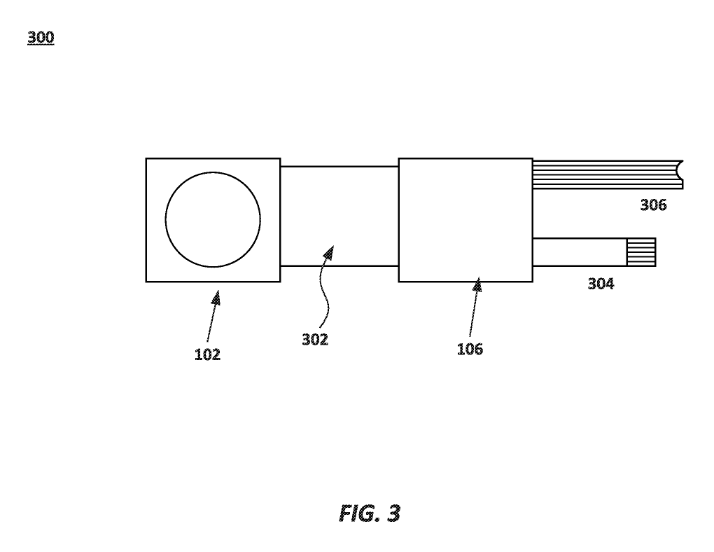

Each of the components, such as the CO sensor 102, the MCU 106, and the button 112 can be connected together using a flex circuit (shown in FIG. 3). The flex circuit can include a flexible connection 204 between MCU 106 and the button 112.

FIG. 3 is a schematic diagram of a flex circuit 300 that includes a carbon monoxide sensor in accordance with embodiments of the present disclosure. The flex circuit 300 can be used to form fit the CO sensor 102 and the microprocessor unit 106 (as well as other units) into the wearable device. The flex circuit 300 includes flexible connection 302 that connects the CO sensor to the MCU 106. The flex circuit 300 also includes a flex connector 304 for connecting the flex circuit 300 to the main PCB 200 of FIG. 2. The flex circuit 300 also includes a harness 306 for connecting other components, such as the speaker, LED, button, etc.

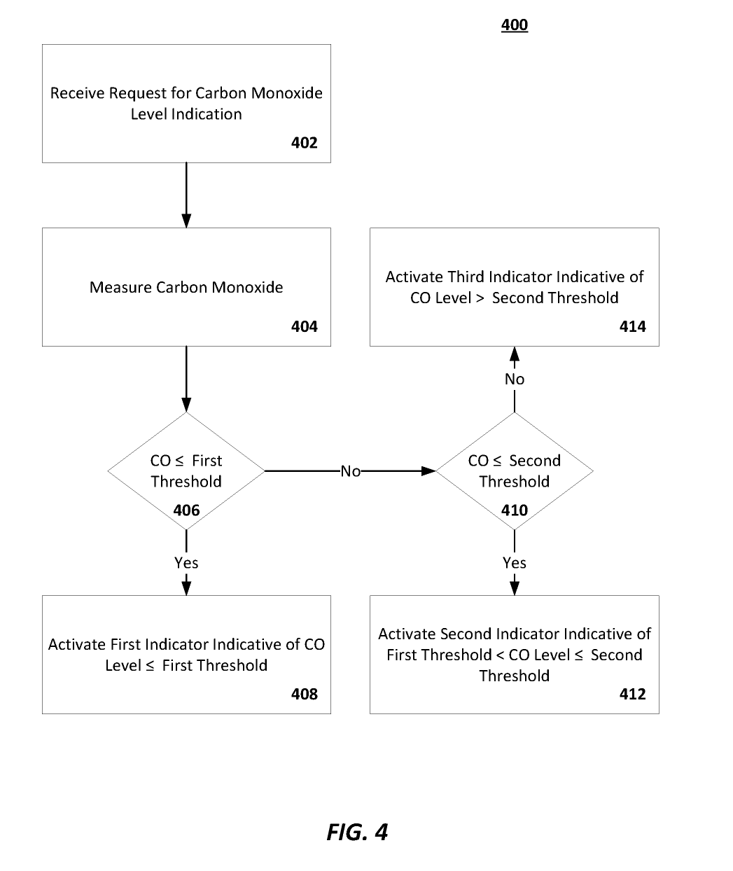

FIG. 4 is a process flow diagram 400 for alerting a wearer of a wearable carbon monoxide exposure limiter of a carbon monoxide level in accordance with embodiments of the present disclosure. A wearable device that includes a CO sensor can receive a request for a carbon monoxide level indication (402). The request can be received based on a timer or on a button press or by other ways. The CO sensor can measure the level of CO in the area (404). The processor can compare the measured level of CO with stored threshold levels (406). If the CO is less than or equal to a first threshold, the wearable device can activate a first indicator indicative of CO levels less than or equal to the first threshold (408). For example, if the CO levels are below the first threshold, the wearable device might indicate a green LED indicating that the area is safe from harmful levels of CO. A recorded message to the same effect can also be played back for the wearer through a speaker.

If, however, the CO level is greater than a first threshold, then the processor can compare the CO level to a second threshold (410). If the CO level is less than or equal to the second threshold, the processor can activate a second indicator indicating a CO level between the first and second threshold (412). For example, the LED can be a yellow color warning of potentially harmful levels of CO. A recorded message to the same effect can also be played back for the wearer through a speaker.

If, however, the CO level is greater than the second threshold, the processor can activate a third indicator indicating a CO level greater than the second threshold (414). For example, a red LED can be activated to indicate a high and dangerous level of CO. A recorded message to the same effect can also be played back for the wearer through a speaker.

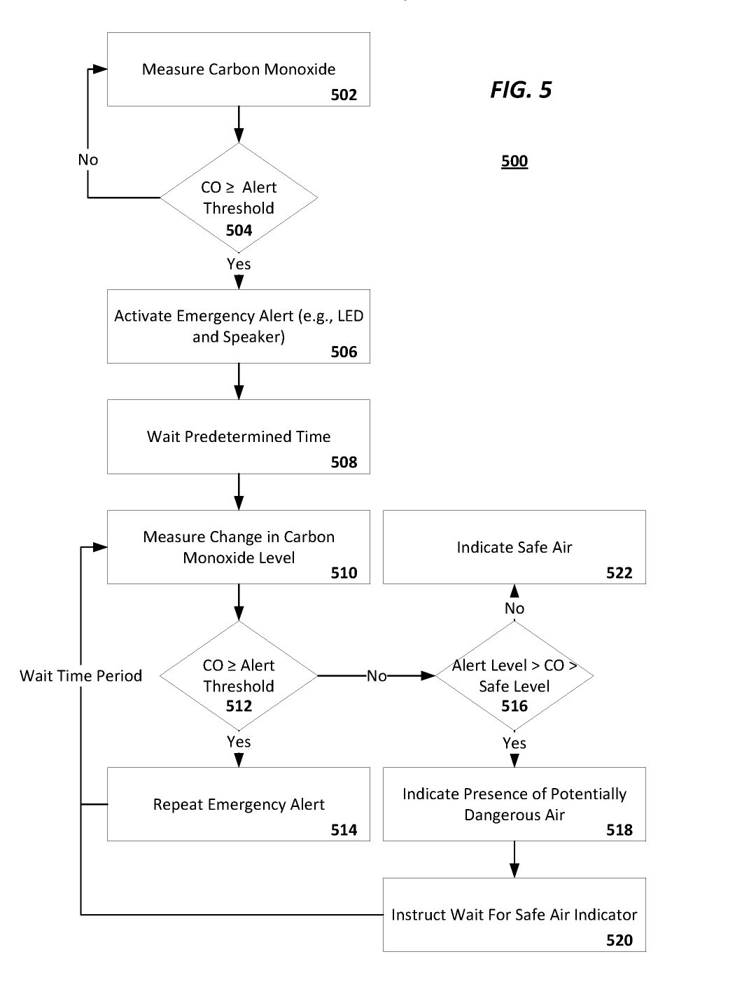

FIG. 5 is a process flow diagram 500 for providing an emergency alerting accordance with embodiments of the present disclosure. This process flow diagram 500 provides a way to alert a wearer of dangerous levels of CO, and also to provide updates on the state of the CO level so that the wearer can take further action if necessary to move to a safer area.

At the outset, a CO level can be measured (502). CO levels can be continuously measured as long as the wearable device has power, at preset times of the day, such as meal cooking times, based on an affirmative request, such as a button press, etc.

The processor can compare the measured CO level to an alert threshold (504). An alert threshold can include a particularly high level of CO that indicates an extremely dangerous environment or emergency level of CO, which would prompt immediate action to move to safer air.

If the CO levels are lower than an alert threshold, the wearable can continue monitoring (502).

If, however, if the CO levels measured are greater than or equal to the alert threshold value, then the wearable can activate an emergency alert (506). The emergency alert can include the activation of LEDs, such as red LEDs, and in a sequence or flashing format; and the emergency alert can include an actual message that prompts the wearer to move to find cleaner air.

The wearable device can wait for a predetermined amount of time before taking another CO measurement (508). The wearable device can then take another CO measurement or can measure a change in CO levels (510). The CO level can be compared to the alert threshold (512). If the levels equal or exceed the alert levels, then the emergency alert can be repeated (514). A time period can be waited again, and the measurement redone (510) until the CO level drops to a safer level.

If, after taking a CO measurement, the CO levels are less than the alert level, the processor can determine whether the measured CO levels are completely safe or if the CO levels can still pose a health risk (though not at emergency levels) (516). If the CO levels are still too high for safety, the processor can indicate that the air is potentially dangerous (518). For example, a yellow LED can indicate moderately dangerous air, as opposed to safe air. An audible recording can also provide that information.

The wearable device can instruct the wearer to wait or to continue trying to find clean air (520). The CO levels are taken again (510) after some predetermined amount of time. If the measured CO levels indicate safe air, then a safe air indicator is provided to the wearer (522). A safe air indicator can include a green LED light and/or a message instructing the wearer of clean air.

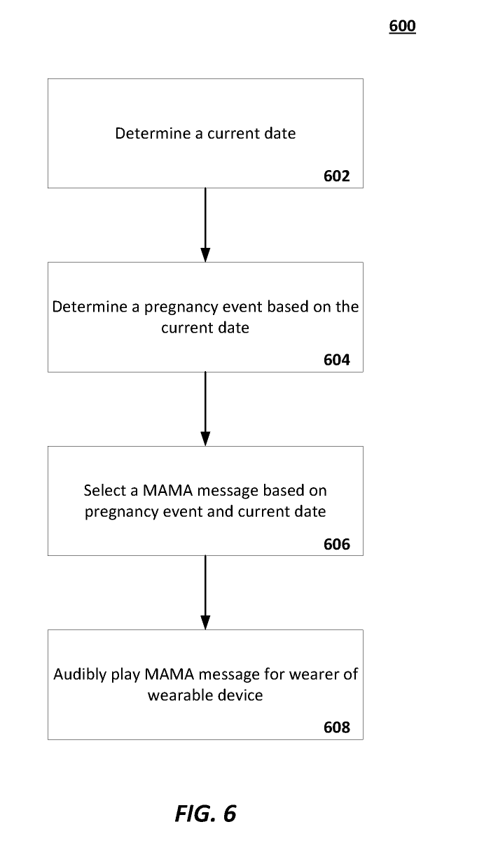

FIG. 6 is a process flow diagram 600 for providing maternity messages to a wearer of a wearable device including a carbon monoxide exposure limiter. A current date can be determined by checking a date programmed into the wearable device (602). A pregnancy event or pregnancy-related information can be determined based on the current date (604). A Mobile Alliance for Maternal Action (MAMA) message can be selected from a plurality of messages. The MAMA message can be selected based on the pregnancy event, the current date, or some combination. A pregnancy event can include a due date, a doctor appointment, etc. The MAMA message can be audibly played for the wearer to remind or alert the wearer of an upcoming or imminent pregnancy-related event or information.

An example of pregnancy-related information can include a notification of a date or milestone, such as “20 weeks of pregnancy.” The maternal wellness wearable can provide the wearer on two specific days of the week, every week, a MAMA message that corresponds to the wearer's current week of pregnancy. Other information can also be provided, such as notifications of when it is time to take medication, or perform a blood test for diabetes suffers, etc.

The following tables provide correlations between threshold CO levels and the indicators associated with various levels of CO.

TABLE 1
User Requests Feedback

| CO | | LED | LED | Audible |
|---|---|---|---|---|
| Characteristic | CO Level (ppm) | Color | Signaling | Alert |
| Safe | 57 > CO | Green | Slow blink | Off |
| Dangerous | 73 ≧ CO ≧ 57 | Yellow | Slow blink | Off |
| Emergency | CO > 73 | Red | Slow blink | Off |
| (Evac) | | | | |

TABLE 2
Instantaneous Exposure Alert

| CO | | LED | LED | Audible |
|---|---|---|---|---|
| Characteristic | CO Level (ppm) | Color | Signaling | Alert |
| Safe | [CO]1 < 200 or | Green | Slow blink | Off |
| | [CO]5 < 800 | | | |
| Emergency | [CO]1 > 400 or | Red | Fast blink | Loud Beep |
| (Evac) | [CO]5 > 800 | | | |

([CO]1 = average CO for 1 min, [CO]5 = average CO for 5 minutes

Example 1 is a wearable device comprising a carbon monoxide detector to detect carbon monoxide; a light emitting diode (LED); a memory for storing data; and a controller to determine that a carbon monoxide (CO) level detected by the carbon monoxide detector exceeds a threshold level; and activate the LED based on the determination that the CO level detected by the CO detector exceeds the threshold level.

Example 2 may include the subject matter of example 1, wherein the memory stores threshold carbon monoxide (CO) levels comprising a first threshold CO level indicating a safe level; and a second threshold CO level indicating an emergency level. In some embodiments, a third threshold level resides between the safe level and the emergency level, and is referred to as an acute level.

Example 3 may include the subject matter of any of examples 1 or 2, wherein the LED is a first LED of a first color, and the wearable device further comprises a second LED of a second color.

Example 4 may include the subject matter of any of examples 1 or 2 or 3, wherein the controller is configured to determine that the CO level is above the safe level; and activate the first LED of the first color based on determining that the CO level is above the safe level.

Example 5 may include the subject matter of any of examples 1 or 2 or 3, wherein the controller is configured to determine that the CO level is above the emergency level; and activate the second LED of the second color based on determining that the CO level is above the emergency level.

Example 6 may include the subject matter of any of examples 1 or 2 or 3 or 4 or 5, wherein the memory further stores a plurality of recorded messages to indicate to a wearer of the wearable device of a carbon monoxide alert.

Example 7 may include the subject matter of example 6, wherein the controller is configured to determine that the CO level is above the safe level; and select a recorded message from the plurality of recorded messages that indicates that the CO level is above the safe level.

Example 8 may include the subject matter of example, wherein the controller is configured to determine that the CO level is above the emergency level; and select a recorded message from the plurality of recorded messages that indicates that the CO level is above the emergency level.

Example 9 may include the subject matter of any of examples 1 or 2 or 3 or 4 or 5 or 6 or 7 or 8, further comprising a speaker to provide an audible recorded message to a wearer of the wearable device based on the CO level detected.

Example 10 may include the subject matter of any of examples 1 or 2 or 3 or 4 or 5 or 6 or 7 or 8, wherein the memory stores pregnancy-related information for a wearer of the wearable device; and a plurality of Mobile Alliance for Maternal Action (MAMA) messages relating to a pregnancy of the wearer, the messages indicating one or more actions to be taken by the wearer.

Example 11 may include the subject matter of example 10, wherein the pregnancy related information comprises dates of events associated with a pregnancy, and wherein the controller is configured to determine a current date; determine an event associated with the current date; and select a MAMA message based on the date and the event.

Example 12 is a method comprising measuring a carbon monoxide level with a carbon monoxide sensor on a wearable device; determining a danger assessment based on the measured level of CO; activating an indicator informing a wearer of the wearable device indicating the danger assessment of the measured CO level.

Example 13 may include the subject matter of example 12, further comprising determining a current date; determining a pregnancy event based on the current date; selecting a Mobile Alliance for Maternal Action (MAMA) message based on the pregnancy event and the current date; and playing the MAMA message audibly.

Example 14 may include the subject matter of any of examples 12 or 13, further comprising determining that the CO level is above a safe level; and activating a first LED of a first color based on determining that the CO level is above the safe level.

Example 15 may include the subject matter of any of examples 12 or 13, further comprising determining that the CO level is above an emergency level; and activating a second LED of a second color based on determining that the CO level is above the emergency level.

Example 16 is a system comprising a wearable device comprising a carbon monoxide detector to detect carbon monoxide; a light emitting diode (LED); a memory for storing data; and a processor. The processor is configured to determine that a carbon monoxide (CO) level detected by the carbon monoxide detector exceeds a threshold level; and activate the LED based on the determination that the CO level detected by the CO detector exceeds the threshold level. The system also includes an ornamental housing, the ornamental housing comprising a housing for the wearable device.

Example 17 may include the subject matter of example 16, wherein the memory stores threshold carbon monoxide (CO) levels comprises a first threshold CO level indicating a safe level; and a second threshold CO level indicating an emergency level.

Example 18 may include the subject matter of any of examples 16 or 17, wherein the LED is a first LED of a first color, and the wearable device further comprises a second LED of a second color.

Example 19 may include the subject matter of example 18, wherein the controller is configured to determine that the CO level is above the safe level; and activate the first LED of the first color based on determining that the CO level is above the safe level.

Example 20 may include the subject matter of example 18, wherein the controller is configured to determine that the CO level is above the emergency level; and activate the second LED of the second color based on determining that the CO level is above the emergency level.

Example 21 may include the subject matter of any of examples 16 or 17 or 18 or 19 or 20, wherein the memory further stores a plurality of recorded messages to indicate to a wearer of the wearable device of a carbon monoxide alert.

Example 22 may include the subject matter of example 21, wherein the controller is configured to determine that the CO level is above the safe level; and select a recorded message from the plurality of recorded messages that indicates that the CO level is above the safe level.

Example 23 may include the subject matter of example 22, wherein the controller is configured to determine that the CO level is above the emergency level; and select a recorded message from the plurality of recorded messages that indicates that the CO level is above the emergency level.

Example 24 may include the subject matter of any of examples 16 or 17 or 18 or 19 or 20 or 21 or 22 or 23, further comprising a speaker to provide an audible recorded message to a wearer of the wearable device based on the CO level detected.

Example 25 may include the subject matter of any of examples 16 or 17 or 18 or 19 or 20 or 21 or 22 or 23 or 24, wherein the memory stores pregnancy-related information for a wearer of the wearable device; and a plurality of Mobile Alliance for Maternal Action (MAMA) messages relating to a pregnancy of the wearer, the messages indicating one or more actions to be taken by the wearer.

Example 26 may include the subject matter of any of examples 1 or 2 or 3 or 4 or 5 or 6 or 7 or 8 or 9 or 10 or 11, and also may include that the wearable device has one or more solar cells for supplying power to the processor, the LEDs, the CO sensor, and the memory.

Example 27 may include the subject matter of any of examples 1 or 2 or 3 or 4 or 5 or 6 or 7 or 8 or 9 or 10 or 11 or 26, wherein the memory also stores medical information, and wherein the memory stores messages associated with the medical information, the medical information including one or more of a vaccine schedule for scheduling a time for taking a vaccine, a therapy schedule, a diseases management schedule, etc.

Example 28 may include the subject matter of any of examples 12 or 13 or 14 or 15 or 16 or 17, further including charging the wearable device by exposing the wearable device to light.

Example 29 may include the subject matter of any of examples 12 or 13 or 14 or 15 or 16 or 17 or 28, further including providing an audible message to the wearer that pertains to a medical schedule, such as a vaccination schedule, a therapy schedule, a diseases management schedule, etc.

Advantages of the present disclosure are readily apparent to those of skill in the art. Among the various advantages of the present disclosure include the following:

Aspects of the present disclosure can provide a carbon monoxide notification to a wearer of a culturally sensitive and culturally relevant wearable device. Additionally, the wearer can be provided with scheduling information related to maternity and other health considerations, such as vaccine schedules.

While this specification contains many specific implementation details, these should not be construed as limitations on the scope of any disclosures or of what may be claimed, but rather as descriptions of features specific to particular embodiments of particular disclosures. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

1. A wearable ornamental device comprising:
a carbon monoxide detector to detect carbon monoxide;
a light emitting diode (LED);
a memory for storing data; and
a controller to:
determine that a carbon monoxide (CO) level detected by the carbon monoxide detector exceeds a threshold level; and
activate the LED based on the determination that the CO level detected by the CO detector exceeds the threshold level;
wherein the memory stores:
pregnancy-related information for a wearer of the wearable ornamental device; and
a plurality of Mobile Alliance for Maternal Action (MAMA) messages relating to a pregnancy of a wearer of the wearable ornamental device, the messages indicating one or more actions to be taken by the wearer; and
the controller to:
identify a pregnancy-related milestone based on the pregnancy-related information stored in memory based on a current date; and
audibly signal one of the MAMA messages to the wearer based on the pregnancy-related milestone.

2. The wearable device of claim 1, wherein the memory stores threshold carbon monoxide (CO) levels comprising:
a first threshold CO level indicating a safe level;
a second threshold CO level indicating an emergency level.

3. The wearable device of any of claims 1, wherein the LED is a first LED of a first color, and the wearable device further comprises a second LED of a second color.

4. The wearable device of claim 3, wherein the controller is configured to:
determine that the CO level is above the safe level; and
activate the first LED of the first color based on determining that the CO level is above the safe level.

5. The wearable device of claim 3, wherein the controller is configured to:
determine that the CO level is above the emergency level; and
activate the second LED of the second color based on determining that the CO level is above the emergency level.

6. The wearable device of claim 1, wherein the memory further stores a plurality of recorded messages to indicate to a wearer of the wearable device of a carbon monoxide alert.

7. The wearable device of claim 6, wherein the controller is configured to:
determine that the CO level is above the safe level; and
select a recorded message from the plurality of recorded messages that indicates that the CO level is above the safe level.

8. The wearable device of claim 6, wherein the controller is configured to:
determine that the CO level is above the emergency level; and
select a recorded message from the plurality of recorded messages that indicates that the CO level is above the emergency level.

9. The wearable device of claim 6, further comprising a speaker to provide an audible recorded message to a wearer of the wearable device based on the CO level detected.

10. (canceled)

11. The wearable device of claim 1, wherein the pregnancy related information comprises dates of events associated with a pregnancy, and wherein the controller is configured to determine a current date; determine an event associated with the current date; and select a MAMA message based on the date and the event.

12-15. (canceled)

16. A system comprising:
a wearable device comprising:
a carbon monoxide detector to detect carbon monoxide;
a light emitting diode (LED);
a memory for storing data; and
a processor to:
determine that a carbon monoxide (CO) level detected by the carbon monoxide detector exceeds a threshold level; and
activate the LED based on the determination that the CO level detected by the CO detector exceeds the threshold level; and
an ornamental housing, the ornamental housing comprising a housing for the wearable device;
wherein the memory stores:
pregnancy-related information for a wearer of the wearable ornamental device; and
a plurality of Mobile Alliance for Maternal Action (MAMA) messages relating to a pregnancy of a wearer of the wearable ornamental device, the messages indicating one or more actions to be taken by the wearer; and
the controller to:
identify a pregnancy-related milestone based on the pregnancy-related information stored in memory based on a current date; and
audibly signal one of the MAMA messages to the wearer based on the pregnancy-related milestone.

17. The system of claim 16, wherein the memory stores threshold carbon monoxide (CO) levels comprising:
a first threshold CO level indicating a safe level;
a second threshold CO level indicating an emergency level.

18. The system of any of claims 16, wherein the LED is a first LED of a first color, and the wearable device further comprises a second LED of a second color.

19. The system of claim 18, wherein the controller is configured to:
determine that the CO level is above the safe level; and
activate the first LED of the first color based on determining that the CO level is above the safe level.

20. The system of claim 18, wherein the controller is configured to:
determine that the CO level is above the emergency level; and
activate the second LED of the second color based on determining that the CO level is above the emergency level.

21. The system of claim 16, wherein the memory further stores a plurality of recorded messages to indicate to a wearer of the wearable device of a carbon monoxide alert.

22. The system of claim 21, wherein the controller is configured to:
determine that the CO level is above the safe level; and
select a recorded message from the plurality of recorded messages that indicates that the CO level is above the safe level.

23. The system of claim 22, wherein the controller is configured to:
determine that the CO level is above the emergency level; and
select a recorded message from the plurality of recorded messages that indicates that the CO level is above the emergency level.

24. The system of claim 22, further comprising a speaker to provide an audible recorded message to a wearer of the wearable device based on the CO level detected.

25. (canceled)
